(19) United States
(12) Patent Application Publication
(10) Pub. No.: US 2021/0128248 A1
(43) Pub. Date: May 6, 2021

(54) ROBOTIC MEDICAL APPARATUS, SYSTEM, AND METHOD

(71) Applicant: AVRA Medical Robotics, Inc., Orlando, FL (US)

(72) Inventors: Barry Cohen, Fort Lauderdale, FL (US); Alexandre Clug, Palm Beach Gardens, FL (US); Eytan Pollak, Oviedo, FL (US); Zhihua Qu, Orlando, FL (US)

(21) Appl. No.: 16/071,311

(22) PCT Filed: Jun. 20, 2017
(86) PCT No.: PCT/US2017/038398

§ 371 Date: Jul. 19, 2018

Related U.S. Application Data
(60) Provisional application No. 62/493,002, filed on Jun. 20, 2016.
(60) Provisional application No. 62/499,965, filed on Feb. 9, 2017.
(60) Provisional application No. 62/499,952, filed on Feb. 9, 2017.
(60) Provisional application No. 62/499,954, filed on Feb. 9, 2017.
(60) Provisional application No. 62/499,971, filed on Feb. 9, 2017.
(60) Provisional application No. 62/499,970, filed on Feb. 9, 2017.

(51) Int. Cl. A61B 34/20; A61B 34/32; A61B 17/3211; A61B 17/3201; A61B 18/14; A61M 37/00; A61B 18/04; A61B 90/00

(52) U.S. Cl. CPC A61B 34/20; A61B 34/32; A61B 17/3211; A61B 17/3201; A61B 2018/00595; A61M 37/0015; A61B 18/042; A61B 90/37; A61B 18/14

(57) ABSTRACT

A robotic system for treating the skin of a patient has a robotic arm with several degrees of freedom supporting a navigation unit at an end distal to its base. The navigation unit holds a medical instrument, such as a scalpel, a microneedle tool, a plasma skin treatment device, or other medical instrument. The navigation unit has sensors that sense the distance and angle of attitude of the tool relative to the patient. A control system provides for a programmed movement of the medical instrument through a series of movements on or near the skin of the patient. Relying on the sensors in the navigational unit, the control system navigation maintains the medical instrument at a predetermined operating distance from and at a predetermined angle to the skin of the patient as the instrument is moved through the procedure, whether controlled robotically and autonomously or manually.

### RELATED APPLICATIONS

This application claims the benefit of U.S. provisional patent application Ser. No. 62/493,002 filed Jun. 20, 2016, U.S. provisional patent application Ser. No. 62/499,952 filed Feb. 9, 2017, U.S. provisional patent application Ser. No. 62/499,954 filed Feb. 9, 2017, U.S. provisional patent application Ser. No. 62/499,965 filed Feb. 9, 2017, U.S. provisional patent application Ser. No. 62/499,970 filed Feb. 9, 2017, and U.S. provisional patent application Ser. No. 62/499,971 filed Feb. 9, 2017.

### FIELD OF THE INVENTION

This invention relates to the field of robotic systems that perform medical procedures using robotically-controlled medical instruments, and more particularly to robotic systems that operate automatically, at least to a degree, without constant human control, and to methods and components for such robotic systems.

### BACKGROUND OF THE INVENTION

Current medical procedures performed by human doctors do not provide consistent and precise control of human-controlled or cellular-level tools, and as a result patients at times experience potentially painful or less than uniform results due to human errors, or due simply to variations in human performance from one medical practitioner to another, or even variation over time for a given practitioner.

Although there are some robotically controlled procedures in use today, they are generally first-generation robotic systems that are prohibitively large and heavy, and also quite expensive, making them unavailable except in a very limited number of facilities.

Current medical procedures also cannot provide consistent and precise control of cellular level tools from a remote location.

### SUMMARY OF THE INVENTION

It is therefore an object of the invention to provide a medical robotic system and method of operation that overcomes the drawbacks of prior art medical procedures and robotic medical systems.

According to an aspect of the invention, a robotic system for treating the skin of a patient comprises a mechanical support device having a support portion. The mechanical support device supports the support portion in a three-dimensional space of three-dimensional locations and in a range of three-dimensional angular orientations. The mechanical support device is configured to move the support portion in the three-dimensional space and over the range of angulations responsive to electronic control. A tool connection is fixedly supported on the support portion of the mechanical support device. A medical tool is supported on the tool connection so as to move with it and with the support portion of the mechanical support device. The medical tool has an operative portion directed in an operative direction and configured to therapeutically or cosmetically interact with the skin of the patient. A sensor apparatus is supported so as to be in a fixed position relative to the medical tool, the sensor apparatus sensing the skin of the patient and generating sensor electrical signals indicative of a position and orientation of the operative portion of the tool relative to a part of the skin of the patient with which the tool is interacting. Navigation electronics receive the sensor electrical signals, and based on them control the mechanical support device. The mechanical support device moves the support portion, tool connector, tool and sensor over the skin of the patient to a series of predetermined locations in each of which the operative portion of the tool interacts with a respective treatment area of the skin of the patient.

According to another aspect of the invention, a method for treating a skin region of a patient comprises scanning the skin region of the patient so as to derive three-dimensional data defining a surface contour of the skin region, and determining a number of points on the skin region at which treatment is to be applied. A robotic apparatus is provided that movably supports a skin treatment tool in a range of positions and angular orientations responsive to electrical control signals, and the skin treatment tool has a sensor apparatus supported fixedly with respect to it so as to move with it. The treatment of the skin region is performed with the skin treatment tool, wherein the skin treatment tool is moved to a number of locations and orientations by the robotic apparatus, and wherein, in each of the locations, an operative effect of the tool is directed to a respective point of the number of points. During the treatment a relative distance and orientation of the tool relative to the skin region of the patient is sensed continually using the sensor apparatus, wherein the sensor apparatus generates electrical signals from which said relative distance and orientation are determined. Using the electrical signals, movement of the robotic apparatus is controlled so that, at each of the number of locations, the skin treatment tool is located and oriented at a distance and an angulation relative to the skin region appropriate for the treatment of the skin region using the skin treatment tool.

According to another aspect of the invention, a medical robot system is provided that is “autonomous” or “semi-autonomous”, as determined by the surgeon, which may be done locally or remotely. The medical robotic system relies on a smart guidance component that provides quality and efficiency of operation, even with remote application. The system has precise computer control of a robotically supported tool through a software and hardware configuration, controlled and directed by the surgeon specialist.

In another aspect of the invention, the robotic system supports a tool that is configured and supported movingly so as to remove skin anomalies such as tattoos, wrinkles, or other unwanted skin nuances. This tool is preferably a plasma/helium device mounted to an articular robotic arm providing a more delicate and precise control than earlier robotically controlled tools or human surgical procedures can provide. The robotic arm is controlled by a reticular activating system comprised in total of the robotic arm and its interface controller, the plasma/helium tool, and additional software. The combination of tools and associated apparatus in a configuration that is combined to provide a variety of surgical procedure capabilities including virtually painless and precise resurfacing of human skin for a number of purposes, both cosmetic as well as medical.

The system preferably has a custom attachable/detachable structure supporting the tool that enables the system to be readily adapted to various types of configurations required for a variety of medical procedures by changing the tool. These procedures include but are not limited to cosmetic surgery and specific precise evasive procedures. The other types of surgery use the same controller and robotic arm, with various medical tool attachments along with specific navigation controls based on the specific medical application.

According to another alternative aspect of the invention, the robotic arm supports and controls movement and operation of a micro-needling tool attachment as part of a robotic surgical system. The surgical tool is employed as part of a collaboration of independent medical equipment and devices used in a variety of surgical procedures, including an innovative procedure for skin tightening, pore tightening, and wrinkle care. When the microneedle tool is applied to the skin, under local or topical anesthesia, sterile micro-needles create many microscopic channels deep into the dermis of the skin, which stimulate the body to produce new collagen. These channels also improve the penetration of creams containing vitamins A and C, which stimulate skin renewal, making the skin appear fresher and younger. The micro-needling tool is mounted to the controlled robotic arm and provides a more delicate and precise control than earlier robotically-controlled tools or human surgical procedures can provide.

According to another aspect of the invention, a control console provides precise remote command for robotic assisted surgery, relating generally to operation where a surgical robot has one or multiple robotic arms that are each enhanced with respective instrument or tool attachments adaptable to both current and possible future instrument evolution. The tools may include basic to complex hardware, such as a scalpel, scissors, electrocautery, micro cameras, and other commonly-used surgical apparatus. The console provides surgeons with very precise control of movement of the remote robot along with 3-D vision, all through the control console.

Furthermore, the system provides precise computer control of a surgical robotic device from a remote location, e.g., by a surgeon in the United States for a patient located in the Germany. This is achieved through a software and hardware configuration controlled and directed by a surgeon specialist.

In one aspect of the invention, a control console is used by a surgeon to delicately and precisely position robotic arms equipped with any number of surgical tool attachments. The remote operations capability ultimately is the same as the surgeon being on-site in the operation theatre. The console enables the surgeon to be accurate and exact in his or her approach to a variety of procedures. The combination of the hardware and software configuration of the system shown, used in conjunction with the control console, mitigates arbitrary quality aberrations.

The remotely-operated system of the invention provides a combination of surgical tools and associated apparatus in a configuration that provides a variety of surgical procedure capabilities remotely. The remote console controls the surgical articular robotic arm remotely, and the console controller is readily adaptable to any surgical robotic configuration that may be required for any of a variety of medical procedures.

According to another aspect of the invention, a surgical mechanical manipulative arm is provided that is compatible with a variety of surgical device attachments from non-unique vendors. This mechanical tool is a precise and highly controllable arm, engineered to accept any of an array of detachable surgical instruments. Its range of angular and spatial movements provides articulation that can meticulously simulate a surgeon's refined human hand movements and medical tool control. As an example, the mechanical arm of the invention can, via attachments and control hardware and software components, provide surgeons with the ability to conduct complex minimally invasive surgical procedures. The precision of its movements makes the mechanical arm of the invention desirable whether the operation is completely autonomous, i.e., completely computer-controlled, partially autonomous, or completely controlled by the surgeon.

The robotic surgical system provides a comprehensive platform, incorporating advanced devices, instrumentation and tools, all driven by linked intelligence and designed by the world's leading robotic surgeons. The system offers a flexibility, mobility, freedom of motion and portability provided by its single or multiple arms, which enable the benefits of minimally invasive surgery to be applicable in all surgical procedures. Portability and lighter weight, with a significantly smaller footprint, enable use of the system of the invention in doctors' offices, group practices, surgical centers or field operations, as well as in hospital operating rooms.

According to another aspect of the invention, a computer system that controls the robot arm and the tool that it controls has a control interface that allows a surgeon, locally or remotely, to review a scan of a patient's body and select a pattern or trajectory of locations on the patient for interaction of the tool on the robotic arm with the patient. The system then, during the operation, relies on sensors on the robotic arm that maintain a desired distance and angulation of the tool relative to the patient.

The system preferably allows for a simulation of a planned procedure, and produces a video for viewing by the supervisor surgeon of the movement of the tool and the robotic arm through the procedure for study and approval before any real procedure is undertaken on the patient in reality. Additionally, the robot arm may be caused to rehearse the operation by actually going through the movements of the procedure in reality without the tool active, and with or without the actual patient being present. The simulation may rely only on a scanned version of the patient while providing real movement of the real robotic arm.

It is also an object of the system of the invention to record all movements of the robotic arm and tool for playback later. This provides for a subsequent review of the procedure, and, where a patient returns for a second procedure, the earlier treatment can be reloaded to provide treatment only in a needed area.

It is also an object of the invention to provide a system in which a library of prior procedures for given treatments is available that a surgeon may select one to implement a planned treatment in an optimal way. The system may recommend a particular stored procedure based on the parameters of the current procedure as well. Ideally, such a collection of prior procedures may be stored and maintained in a cloud-based memory containing earlier procedures performed by experts.

Other objects and advantages of the invention will become apparent from the specification herein.

### BRIEF DESCRIPTION OF THE DRAWINGS

FIG. 1 is a perspective view of a robotic arm, navigation unit and tool of the present invention.

FIG. 2 is a diagram of the components of the present invention.

FIG. 3 is a bottom view of a housing of a navigation unit of the invention.

FIG. 4 is a perspective view of the housing of FIG. 3.

FIG. 5 is a perspective view of a medical tool, camera and sensor assembly to be inserted in the end of the navigation unit housing of FIGS. 3 and 4.

FIG. 6 is a perspective view of the end wall assembly supporting the sensors, tool and camera shown in FIG. 5.

FIG. 7 is a perspective view of a microneedle tool that may be used in the system of the invention.

FIG. 8 is detail view of the operative end of the tool of FIG. 7.

FIG. 9 is a perspective view of an human interface input device for controlling a system according to the invention.

FIG. 10 is a view of a display of the monitor and control system according to the invention with an exemplary screenshot thereon.

FIG. 11 is a flowchart of the set-up of an operation in a system according to the invention.

FIG. 12 is a diagram of a control loop for an initial simulation of movement of the robotic arm of the invention in a planned operation.

FIG. 13 shows a control loop for control of the robotic arm during the operation.

FIG. 14 is a diagram of the movement of the navigation unit and tool of the invention across the skin of a patient.

### DETAILED DESCRIPTION

The surgical robotic system of the invention is a modular construction that offers a portable lightweight and maneuverable robotic solution not previously available in any system in hospitals or surgery centers. The Surgical Robotics System described here provides for robotically-controlled Minimally Invasive Surgical (MIS) systems, and offers an adaptable platform with modular design, size and compelling cost comparisons (system, service and tools).

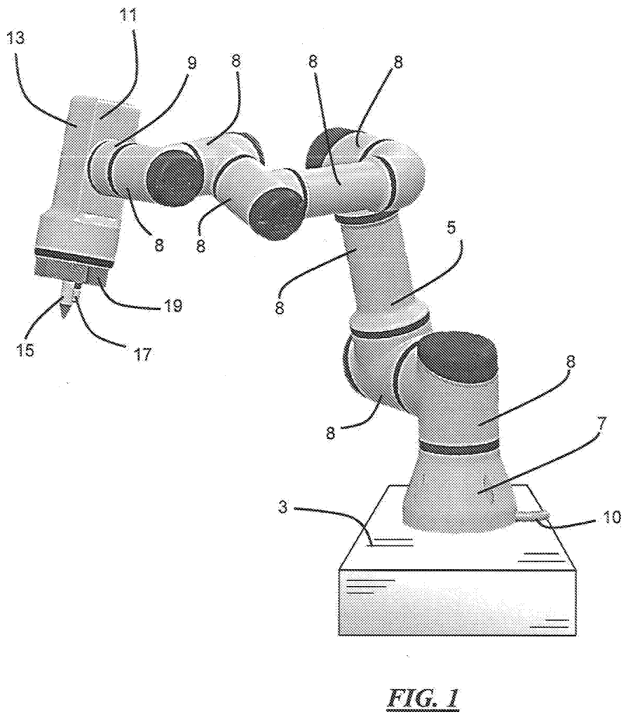

Referring to FIG. 1, a system according to the invention comprises a mounting base 3 fixedly secured to the floor or other vibration-free surface of a building in which the system is employed. The base 3 is normally stationary, but may itself be supported on a track (not shown) that allows it to move to access different locations in the facility.

Robotic Device

The system includes a self-movable mechanical support device in the form of robotic arm 5 with a proximal end 7 that is mounted on base 3. The arm 5 extends through a number of electromechanically movable segments 8 to a distal end or support portion 9. Movement of the arm 5 is directed by electrical signals and power provided via cable 10 from computerized control electronics, not shown. A computerized control system provides control of the arm 5, and the control system includes a computer system with data processing circuitry and data storage, a display configured to display information to a user, and a keyboard and a mouse, and preferably a joystick, for input from a user, as is well known in the art.

Arm 5 has a range of movement such that the arm 5 can selectively move distal end 9 to almost any location and any angular orientation in a three-dimensional space volume around proximal end 7 of the arm 5. Arm 5 preferably has a reach of at least 19.7 inches (0.5 m) from the base 3, and can support a payload of at least 4.4 pounds (2 kg), and preferably 6.6 pounds (3 kg) on support portion 9.

Arm 5 preferably has six degrees of freedom of movement or more, and each of the joints of segments 8 preferably can rotate through a full 360 degrees of rotation, with a speed of rotation of at least 180 degrees per second and, more preferably, at least 360 degrees per second, and is capable of moving the distal end support portion 9 at a speed of at least 39.4 inches per second (1.0 m/sec).

The arm 5 is preferably a digitalized solid-state modular robotic arm. The rotations of the articulated segments 8 are preferably achieved using direct drive, i.e., no cables or pulleys. This provides for an exceptional degree of precision and accuracy, such as that required in specialties such as neurosurgery. In terms of precision of movement, the arm preferably has repeatable accuracy of +/−0.004 inches (+/−0.1 mm). Expressed somewhat differently, arm 5 operates at a tolerance that it can position the tool supported on the distal end 9 with an accuracy within the range of +/−0.009 inches (0.23 mm), and preferably with an accuracy in the range of approximately +/−0.002 inches (0.05 mm), in terms of the precise location of the tool.

Although a variety of robotic arms or other configurations of self-moving mechanical support systems can be used in a robotic system according to the invention, one robotic arm that has been used effectively in the system of the invention is the robot arm sold with the model name UR3 by Universal Robots A/S, a company having a business at Energivej 25, DK-5260 Odense S, Denmark.

Another source of a robotic arm suitable for the present application is the robot arm, with six-degrees of freedom sold by Roboteurs, Inc., through its website www.roboteurs.com.

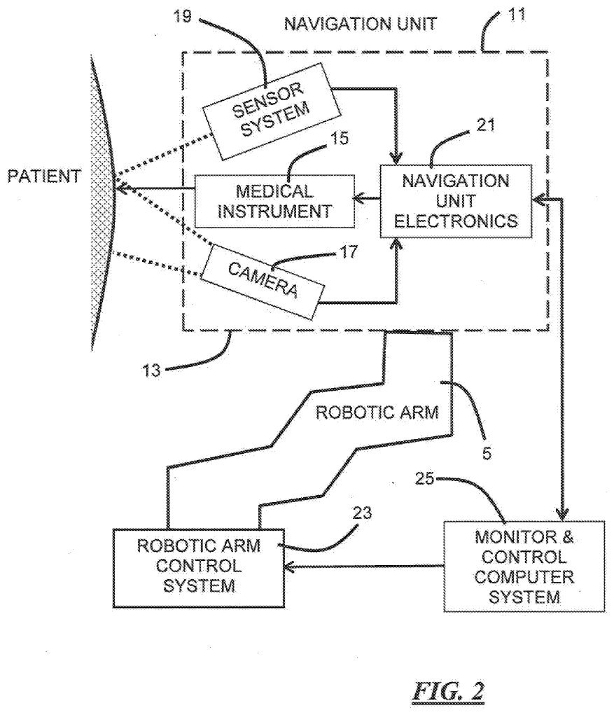

The movement of the robotic arm 5 is controlled by controller electronics in the robotic arm control system 23 (FIG. 2), as will be described below.

Medical Tools and Implements

Referring again to FIG. 1, support portion of distal end 9 of arm 5 has a navigation and connection unit 11 mounted on it. Navigation and connection unit 11 supports inside of its housing 13, a tool 15, a camera 17, and a sensor system or cluster 19.

A variety of medical or surgical instruments or implements may be used as tool 15, which may range from basic to complex hardware, such as a scalpel, scissors, electrocautery, micro cameras, lasers and other commonly-used surgical apparatus.

The preferred embodiment shown employs a plasma-flame skin treatment medical tool used as the tool 15 attached to the robotic arm, and the system does employ such a tool advantageously for various skin treatment procedures, but this should not be seen as a limiting definition of the tool used in the invention.

Particularly preferred as a plasma-flame medical tool is a Bovie laparoscopic J-Plasma tool, sold by the Bovie Medical Corporation of 4 Manhattanville Road, Purchase, N.Y. 10577. The J-Plasma tool has a retractable cutting feature that is used for soft tissue coagulation and cutting during surgery. The system works by passing an inert gas, such as helium, over a uniquely designed blade and energizing the gas to a plasma stream. The distinctive blade design provides the option of retracting or extending the surgical blade, providing multiple modes of operation in a single instrument. Other plasma-flame tools with different configurations may be similarly used.

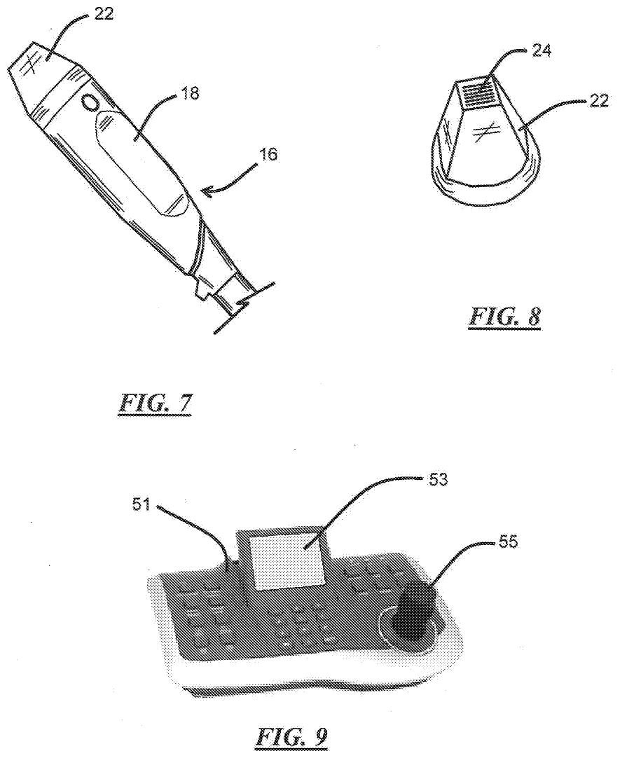

Another tool that may be used advantageously as the tool 15 supported on the robotic arm 5 is the Vivace fractional microneedle tool sold by Aesthetics Biomedical, Inc., 4602 N. 16th Street, Suite 300, Phoenix, Ariz. 85016. This tool is generally illustrated in FIGS. 7 and 8, which show its general outer configuration as a tool and the operative tool surface.

Referring to FIG. 8, the microneedling tool 16 has a generally cylindrical body that can be supported secured fixedly in the navigation unit 11, potentially using an adaptor configured to accommodate the microneedling tool exactly, similarly to the plasma tool, which also has a generally cylindrical body. The operative end 22 of the microneedling tool 16 in the preferred embodiment has a 6×6 array of microneedles, generally indicated at 24, each microneedle having a diameter of about 0.012 inches (0.3 mm). The microneedles are moved during operation to briefly extend out of the operative end 22 of the tool 16 and enter into the skin of the patient being treated to varying depths determined by the surgeon. Special golden and partly insulated microneedles target the dermis without epidermal damage.

In order to press the microneedle tool against the body of the patient, the navigation unit preferably has an electromechanical deployment system inside of the housing 13 that includes a selectively movable holder that supports the microneedle tool and can be selectively activated, such as by a linear solenoid, to extend the tool outward of the housing 13 so as to engage the microneedle matrix against the patient's skin and to activate the needles so that they extend into the patient's skin for treatment. The both functions that can be activated automatically as part of the treatment using the arm 5.

The microneedling tool is used under local or topical anesthesia, and, when applied to the skin, is used to create microscopic channels deep into the dermis, which stimulate the body to produce new collagen. These channels also improve the penetration of vitamins A and C creams which stimulate skin renewal, thereby making the skin appear fresher and younger. The microneedle tool provides 1 MHz/2 MHz precise RF-energy-emitting microneedle electrodes that deliver directly into the dermis, resulting in production of new collagen and elastin, and a minimally invasive dermal volumetric rejuvenation system.

A micromemory motor needling reduces pain and any adverse effects, and the tool has a program-saving function for the various parameters of the treatment. The tool also includes red and blue light-emitting diode (LED) lights that aid skin activity from the treatment.

The weight of this microneedle tool may be substantial and require that the robotic arm 5 have an increased weight capacity, i.e., to support as much as 55 pounds (25 kg) in order to support the microneedle tool, absent redesign of the microneedle tool to reduce the weight of the system for an application such as the present robotic arm system.

Whatever type of tool is used, the tool 15 and the navigation unit and sensor system together form an end effector that places a module at the end of the robotic arm 5 that aids guiding the movement of the arm 5 and operation of the tool 15 through the treatment that is given to a patient in a given procedure, as will be expanded upon herein.

Overall System Configuration

FIG. 2 illustrates the interconnection of the components of the system. Robotic arm 5 supports on it the navigation unit 11, which carries in it a medical instrument 15 and a sensor system 19. Medical instrument or tool 15 has an operative portion that acts on the skin or near under-skin surface tissue of a patient. The sensor system 19 continually, i.e., continuously or repeatedly with a duty cycle that is short, such as for example polling the sensors every 0.10 seconds or less, detects the relative position and angular orientation of the surface of the patient's skin relative to the instrument 15 and the navigation unit 11. The sensor system 19 transmits electrical signals derived from this detection process to navigation unit electronics 21 supported in the housing 13 of the navigation unit 11. Camera 17 also transmits electrical signals that constitute high-definition video of the operational area of the tool 15 to the navigation unit electronics 21.

The navigation unit electronics 21 receives the electrical signals from sensor system 19 and the video signals from camera 17 and transmits them to monitor and control computer system 25. The sensor signals may be transmitted directly as received, or the navigational unit electronics 21 may alternatively include data processing circuitry that, based on the sensor electrical signals, makes a determination of the specific three-dimensional location and angular orientation of the instrument 15 relative to the skin area of the patient being treated by the tool 15, and transmits those electrical signals to the monitor and computer control system 25.

Monitor and Control System

Administration and control of the entire operation by a human surgeon or other specialist or user is provided using monitor and control system 25.

Monitor and control system 25 includes an operator or surgeon console computer system that includes a computer with a processor, electronic memory and data storage, as well as a display screen and keyboard, mouse and joystick input devices that enable the surgeon or other human user to set up the operation and monitor the treatment of the patient while it is proceeding, with a facility for intervening with input at the monitor and control system 25 if desired during the operation, as will be discussed below.

The monitor and control computer system is connected electrically with the navigation unit 11 and receives electrical signals comprising video from the camera 17 and signals containing data from the sensor apparatus 19, which may be raw data or data derived from raw sensor data.

The video from the camera 17 is selectively displayed to a user surgeon on a display device, such as a computer monitor, at the monitor and control system 25. The data from the sensor system 19 is used by the monitor and control system 25 to send electrical signals to robot arm control electronics 23 to cause the robot arm 5 to move in a way that is determined by the monitor and control system 25.

In the preferred embodiment, the robot arm 5 has six separately electromechanically controlled joints that each has a respective motor that rotates that particular joint. The monitor and control system 25 transmits electrical signals that comprise arm command data to robot arm control electronics 23. The command data defines a set of six torque values, each of which has been calculated for a respective one of the rotating joints of the arm 5. Control electronics 23, based on each of the torque values so defined, cause the corresponding joint motor to apply the amount of torque defined in the arm command data for that joint motor, which causes the joint to move in the commanded way.

Navigation Unit

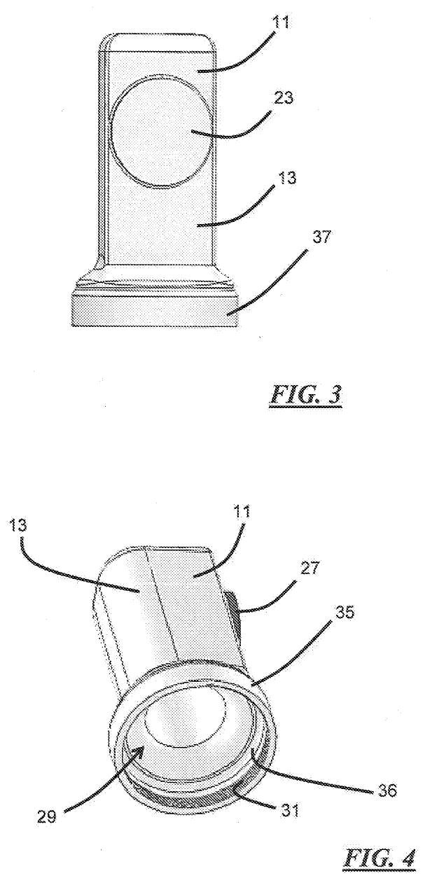

Referring to FIG. 3, the navigation unit includes a housing 13, which has an interior space that receives and supports the components. Housing 13 includes a generally circular connection portion 27 that secures it in engagement with support portion 9 of the arm 5 so as to fixedly connect the navigation unit 11 on the end 9 of the arm 5.

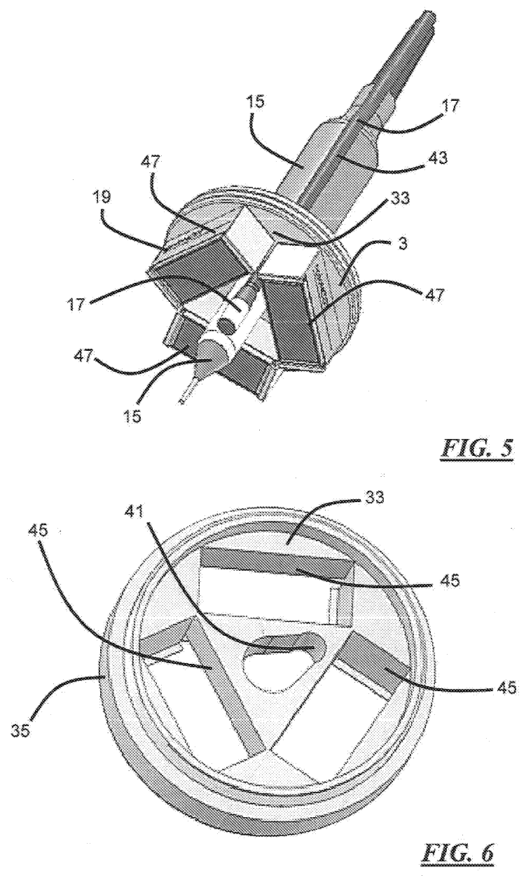

Referring to FIG. 4, the navigation unit 11 has an interior space 29 accessible through a flared opening 31 at the end of the housing 13. The opening 31 receives therein a plate structure 33 seen in FIGS. 5 and 6 that supports the tool 15, the camera 17 and the sensors of the sensor system 19. Opening 31 provides access to annular ledge 36, which engages and supports plate structure 35 against it, and the plate 33 is secured in the housing 13 by threaded retention collar 37 that is threadingly secured around the outer end of the housing 13 and extends around and secures plate 35 on housing 13.

Referring to FIG. 6, the plate 35 has a central aperture 41 configured to receive therein the cylindrical body of tool or instrument 15, and, extending adjacent alongside it, a smaller cylindrical cable 43 of camera 17, which is held in the aperture adjacent to the tool 15 and supports the camera 17 directed to the working area of the tool on the skin of the patient.

Camera 41 is ideally a high-definition video camera, well known in the art, that takes continuous high-definition video of the operational area on the skin of the patient on which the operation of the tool 15 is being directed. This video is transmitted to the monitor and control system 25 via cable 43, where the video may be viewed by the surgeon or specialist monitoring the procedure.

Particularly preferably, camera 17 is a binocular camera that transmits two videos simultaneously from two laterally spaced viewpoints, enabling three-dimensional location of objects, such as markers on the patient, by a computer-vision method for purposes of registering a starting location of the navigation unit 11, as will be described below.

Plate 33 also has three rotationally displaced rectangular apertures 45 that are configured to receive three sensor units 47 of the sensor system 19. The sensors 47 are held each in a respective aperture 45 with the sensing sides thereof directed toward the patient so as to detect the distance of each sensor 47 from the patient's skin in the area of the tool operation. The three distance readings define the relative distance of the tool 15 from the skin, and also define the relative angulation or orientation, or angle of attitude, of the tool relative to the skin.

The distance measurements of each sensor 47 are transmitted to navigation unit circuitry that processes that data and transmits it to the control system 23 so that the control system 25 can position the arm 5 and support portion 9 during the treatment of the patient with the tool 15 is at an appropriate distance and at an appropriate attitude angle, e.g., normal to the skin surface of the patient, for the treatment.

The sensor units 47 are each preferably a distance sensor that uses a laser to determine range to an object with a high degree of accuracy, e.g., by triangulation. Sensors for use in the present invention preferably have a distance measurement accuracy of at least about +/−8 microns, and repeatability as accurate as about 1 micron, or 0.5 microns. Sensors suitable for this system include the red- or blue-laser-based sensors sold under the model name optoNCDT by the Micro-Epsilon Company, whose USA Headquarters is located at 8120 Brownleigh Drive, Raleigh, N.C.

Suitable sensors for use in the navigation unit 11 may also be obtained from the Keyence Corporation of America, located at 500 Park Boulevard, Suite 200, Itasca, Ill. 60143.

The cables and wiring from the navigation unit 11 to the robotic control system 23 and the monitor and control station 25 preferably extend through a passageway internal to the arm 5 to avoid clutter outside the arm 5, and then extend to the robot control system and the monitor and control unit from the base 3 of the system. Any hoses or power cords, etc. for the tool 15 preferably extend through the same passageway in the robot arm.

System Operation

As set out above, overall administration and control of operations using the robot arm 5 with the end effector tool 15 and navigation unit 11 is from the computerized monitor and control system 25, which is usually a surgeon console provided with a display for the supervisory user or surgeon, as well as input devices that the user or surgeon uses to set up an operation and monitor and control the operation while it proceeds. The navigation system 21 and software offers additional accuracy and safety when coupled with the computer console of the monitoring and control system, which incorporates a human-machine interface (HMI) that utilizes the latest tele-manipulation technology.

FIG. 9 shows an interface device that may be provided with the monitor and control system 25 for a user to employ. The device 51 has a number of keys with specific functions corresponding to inputs that may be made by the surgeon or user, as will be discussed below. The device 51 may also have a small monitor screen display 53 that shows information regarding the system or video from the camera 17 on the navigation unit 11. The device 51 also has a joystick 55 that the surgeon may use to directly control the associated tool 15 when the system is in only partially autonomous mode, or when the surgeon assumes full manual control of the system operation.

Alternatively, some of the functions of interface 51 may be emulated in a display screen GUI and activated using a computer mouse attached to the console.

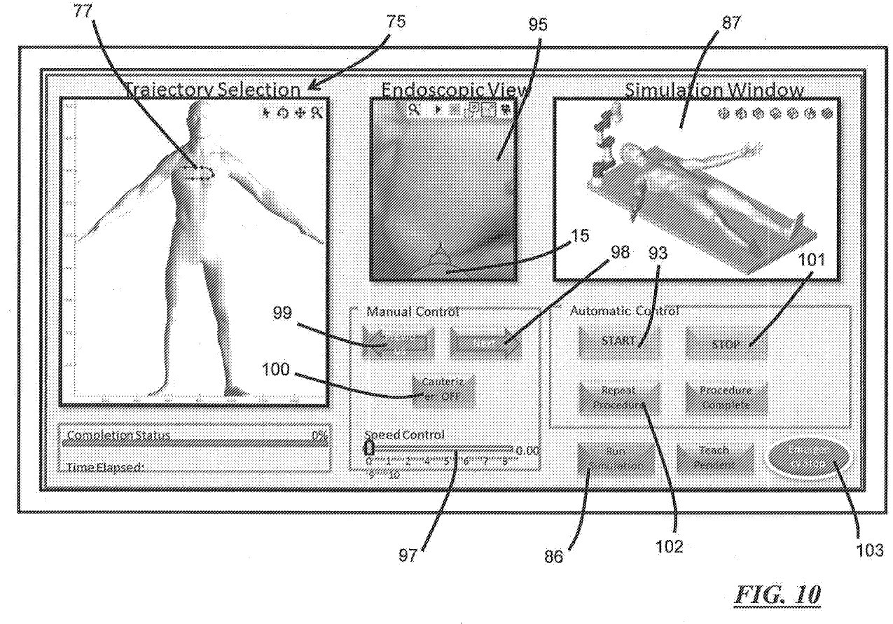

Preferably, the interface device 51 is used together with a full sized monitor, illustrated in FIG. 10, at a surgeon's console of the monitor and control system 25. In the monitor and control system 25, the sensor device outputs and control surgeon's console merges the high-resolution view of the surgical field from camera 17 with the data and information management and action of the robotic system to the biomedical and IT environment in the operating theater. The computer monitor together with the interface device instrument 51 provides a human-machine-interface (“HMI”) for the maneuvering of both the instrument 15 (or instruments if two or more arms are used in the system) and the camera-head 17.

In the preferred embodiment, the robotic system of the invention provides a mechanism to navigate the robotic arm with the surgical instrument autonomously or robotically, meaning without human control, or with only partial limited human control.

Built-in intelligence including sophisticated data analysis and processing, error avoidance, fault tolerance and vital patient information, provide the ability to model, plan and implement customized surgical strategies. In the autonomous or robotic operation of the system, the surgeon is given access by the system to the knowledge, experience, judgment and techniques of the world's master robotic surgeons. The autonomous operation may be pre-programmed based on emulation of the techniques of master human surgeons, as well.

Setup of Operation

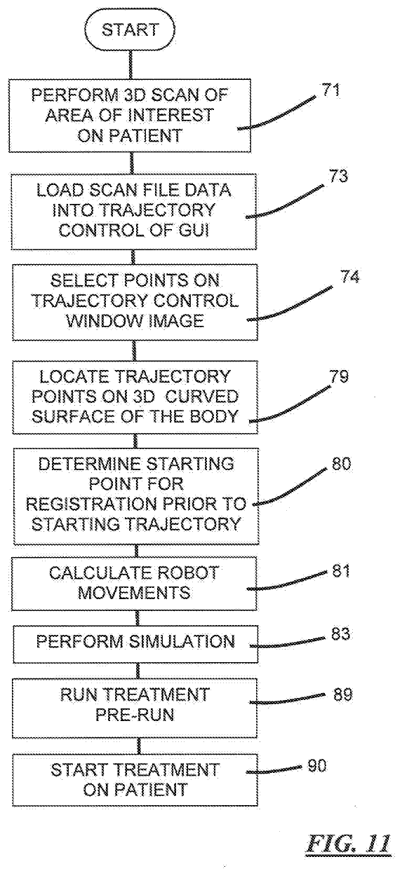

The autonomous mechanism operation is set-up initially by the surgeon using the display interface shown in FIG. 10, and setup proceeds generally as illustrated in the flowchart of FIG. 11.

According to the method of the preferred embodiment, the area of interest to be treated in the patient is determined and subjected to a 3D scan by a 3-D body or surface scanner, as is well known in the art, in step 71. The resulting scan data is transmitted in step 73 to the control system 25. The control system 25 displays an image of the patient with the scanned skin area in the GUI displayed on the control system monitor, as seen in the exemplary screen shot shown in FIG. 10, where the image is displayed in the “Trajectory Selection” window 75 on the screen. The image displayed may be of a scan or photograph of the entire patient's body or a rendered version of the entire body, or just of an area of the patient's skin that is of interest, e.g., a region of skin around a tattoo to be removed.

When the image of the scanned area is shown on the GUI (FIG. 10), the surgeon then selects the series of locations to which the robot arm 5 will move the tool during the treatment or operation in step 74. This can be done by the surgeon at the control console by clicking on the image in window 75 at specific locations so as to identify the points to which the tool 15 of the system should proceed during the treatment, and also defining a duration for the tool to go to all of the set of points so defined. The specific points of the trajectory may be entered by the surgeon at the console, or they may be generated based on trajectories recovered from previous treatment records or other recorded data, such as trajectory patterns employed by experts and stored so as to be accessible to the surgeon console, either locally or remotely, e.g., in the cloud.

The trajectory is defined by trajectory data stored on the control system 25, which trajectory data includes data defining the points or locations of the trajectory, preferably defined in a three-dimensional Cartesian coordinate system of the scan preferably modified by linking it to the location of the robot arm 5. Each point in the trajectory includes a point location on the scanned surface of the patient to which the tool is to go in the operation or treatment of the patient.

The trajectory data also may include data defining a duration specified at setup by the surgeon for the tool to complete its travel to all of the points of the trajectory.

The trajectory data may also include data causing an action to be taken by activation of some function of the tool. For example, where a microneedle tool is used, the tool is moved by the robot arm to a starting point above the treatment area defined by the trajectory point, and then the treatment process is performed, involving activation of a deployment system, preferably electromechanical, that supports the tool 15 and when activated, extends the tool 15 from the navigation unit 11, moving the tool down to the patient to a location where the matrix of needles is just above or abuts the surface of the patient's skin, and then activates the tool to extend the needles into the skin and to apply, if desired, some electromagnetic aspect of the treatment. When treatment is completed, the same system retracts the needles and withdraws the tool 15 back to its starting point in the housing 13 of the navigational unit 11.

The points may be arranged in a trajectory that takes the form of a curved path 77, as seen in FIG. 10, or the trajectory may be a series of points in a line, in a curve, or in a grid pattern, or may even be a series of less linked points that is less like a sequentially-organized path and more like a random point pattern in the area to be treated. For the purposes of this disclosure, it should be understood that the term “trajectory” as used herein applies to any series of points, no matter how geometrically dissociated, and need not be limited to a path of physically sequentially adjacent points in a row or line.

In some cases, of course, as with a plasma-flame tool, the trajectory may be a continuous path that the tool follows at a more or less constant rate that is defined by the surgeon at the console by the definition of the duration of the treatment. In that case, the trajectory does constitute a continuous path, although it may be understood that, in parts of the path predetermined by the surgeon, the control system may be directed to automatically turn off the plasma flame because treatment in those intermediate areas may not be necessary.

Once the trajectory points are identified in the display and entered by the surgeon through the GUI in step 79 (FIG. 11), the trajectory points are then located using the scan data to be specific three-dimensional coordinate locations on the scanned three-dimensional curvature of the patient's body in the area of the trajectory points at step 80.

The scan of the portion of the patient's body is normally defined as a smaller volume unrelated to the space around the robot arm 5. Prior to any robotic arm movement, it is therefore necessary to register the position and orientation of the scanned surface portion of the patient relative to the robot arm 5 itself, so as to provide coordinates of all the points of the treatment in a coordinate system that can be used to control the robot arm movement.

In the preferred embodiment, the relative position of the patient operation area to the robotic arm 5 is determined by registering the location of the patient using a stereoscopic or binocular camera 17 in the robotic arm 5. First, the surgeon places marks, such as two points, on the patient in the area of the operation, usually corresponding to the first two points of the trajectory to be followed, or possibly constituting the entire line of the trajectory. The robotic arm is then moved by the surgeon so that the binocular camera 17 can see markings made on the patient in the operational surface, which it locates in three dimensions by its stereoscopy. Once there is visual detection of those registering marks or points, the relative position of the robot arm 5 to the patient operational area is known to the control system 25, and the kinematics of the robot operation in the procedure to be performed can be calculated.

Alternatively, the scan of the patient may define a large enough area or objects in the robot arm working space such that the Cartesian coordinate system of the scan can be readily converted to a Cartesian coordinate system of the robot arm 5 without the need for registration, optical or otherwise.

Once the locations of the trajectory points in a coordinate system of the robot arm 5 are determined, a simulation is performed, in which an inverse kinetics calculation is employed to rehearse the movements of robot arm 5 to take in moving the navigation unit 11 going through the trajectory points (step 81). The calculation of the movements is also made based on the specific distance from the patient that the tool should be located during the treatment, as well as with the constraint for most tools used with the robotic arm of the invention, that the tool should be directed in an attitude vector that is normal to the patient's skin at each trajectory point, or at some other predetermined appropriate angle of attitude relative to the surface of the patient's skin in the relevant area. These calculations of orientation and distance of the tool in the pre-op simulation are made purely based on the 3D contours of the scanned portion of the patient.

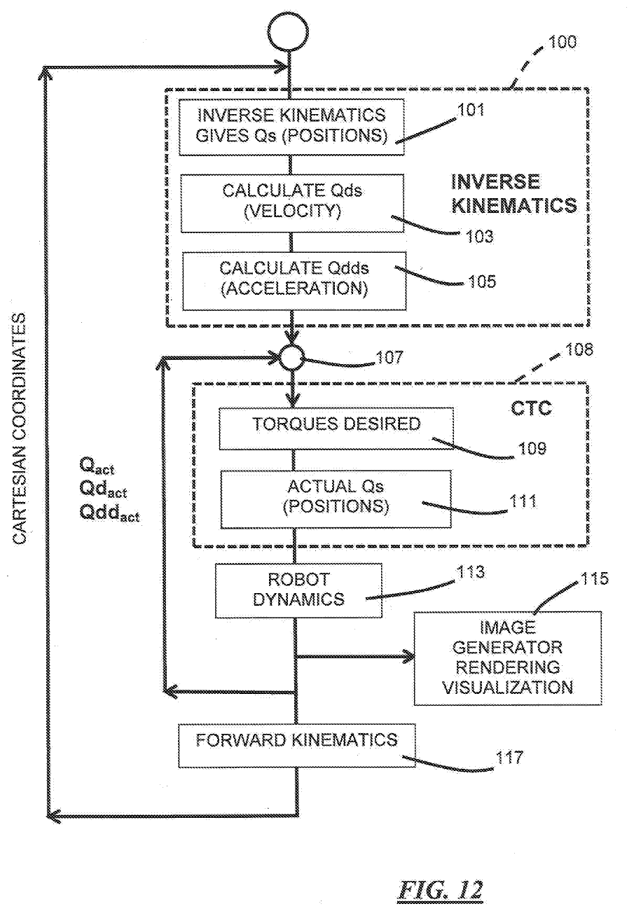

The process for simulating the movements of the robotic arm 5 before performing the actual operation is obtained by the control loop shown in FIG. 12.

The initial trajectory point and desired speed of travel through the trajectory are provided in Cartesian coordinates to a program on the control system 25 that applies the inverse kinematics determination 100 that determines from the desired position and orientation of the tool 15 in the navigation unit 11 the desired Q values for the arm, meaning the desired angular position of the joints of virtual the robot arm of the simulation, in step 101. The desired angular velocity Qds and the desired angular acceleration Qdds of each joint are also calculated (steps 103 and 105). Those values are sent to comparator 107, where data defining the current actual values of the angular position, velocity and acceleration (Q_{act}, Qd_{act} and Qdd_{act}) of the joints of the virtual robot arm are subtracted from the desired values. Data representing the determined difference is then sent to a CTC program 108 running on the control system 25, which determines desired torques to be applied in the arm (step 109) as well as the actual positions of the joints of the arm 5 (step 111). CTC program 108 also includes a known method of control-loop damping, e.g., using a PID or PD controller or something analogous, to prevent jitter of the tool or other typical control-loop problems.

The resulting torque and position values are sent to a robot dynamics simulation program 113. This program 113 determines the simulated outcome in terms of the rotational positions, speed and acceleration of each of the joints of the robot arm 5. That data can be shown to the surgeon as the simulation proceeds by 3D modeling the robot arm and the patient's body or a portion of it in a three-dimensional virtual environment, and rendering sequential two-dimensional images of the progressive views of the virtual robot arm and patient using an image generator, which renders video imagery showing the position of the arm in a simulated view, as is well known in the art. The data of the virtual robot arm position and movement in the computer model is also looped back as the current Q_{act}, Qd_{act} and Qdd_{act} values to be applied to comparator 107, as well as being transmitted to a Forward Kinematics program 117, where the angles of the robot arm parts are converted to Cartesian coordinates for locations and directional vectors. Those Cartesian coordinates of the position and movement of the robotic arm 5 are used to determine when the robot arm has reached a given point in the trajectory that it is processing, which, when reached is replaced by the next point in the trajectory until the simulation reaches the final point and ends.

The surgeon reviews the simulation of the procedure to be performed by pressing the virtual GUI button 86 labeled “Run Simulation” to cause the system 25 to execute the robot commands in simulation, where the control system uses the 3D virtual model of the arm 5 and an exemplary 3D model of the patient to preview the operation to be performed (step 83). In that model, the patient remains stationary and the virtual robot arm moves substantially as it would in reality. The video rendered in 3D from the model of the patient and the robot arm by the image generator operating on the control system 25 is presented at the surgeon console display GUI at the sub-screen GUI visualizer area 87 labeled “Simulation Window” for review by the surgeon.

If the video simulation indicates that the proposed operation of the robot arm 5 is acceptable, the surgeon may elect to further run a surgery pre-run (step 89), in which the robot arm 5 and navigation unit 11 and tool 15 are actually physically run through the procedure without the tool 15 being active.

Treatment Procedure

When the surgeon is satisfied with the trajectory and the procedure employing it, the surgeon then initiates the actual operation on the patient with the tool 15 active (step 91) by clicking on the “Start” virtual button 93 in the GUI. The system then executes the defined procedure (step 90) and the arm 5 moves the End Effector through the trajectory points as defined at the rate specified by the speed control, as described below.

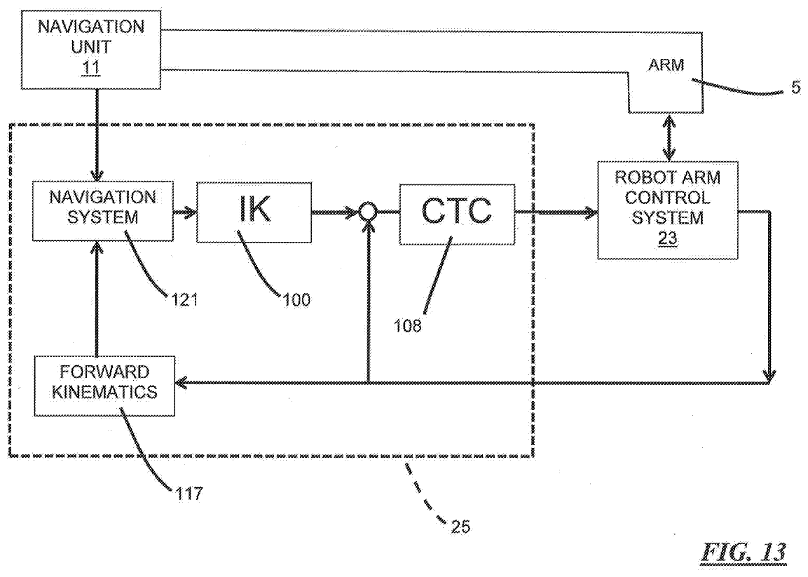

FIG. 13 shows the control loop for the execution of the operation. The control loop shown shares some of the software programs that are used with the initial simulation of the procedure, i.e., inverse kinematics module 100, comparator 107, and CTC module 108, all of which are software-implemented program modules that run on the control system 25. Navigation system 121 is also a program module running on control system 25, and it administers the progress of the tool 15 over the predefined trajectory of locations.

Navigation system 121 has access to data storage on system 25 that defines all the trajectory location coordinates. In addition, navigation system 121 continually receives on a short duty cycle repeated outputs of the sensor data or other data defining the direction of orientation of the tool and its distance from the patient from sensors 19, 47 of the navigation unit 11. Using that data, navigation system 121 determines a current desired location and orientation for the tool 15 in Cartesian coordinates, i.e., tool location and tool-direction vector.

The navigation system 121 determines the desired position of the tool on the robot arm based on two primary parameters or considerations:
1. the distance and orientation of the tool from the patient as detected by the sensors apparatus of the navigation unit should be maintained; and
2. the tool should be moved through the points of the trajectory at a speed so as to arrive at the final trajectory point within the specified duration of the procedure.

Generally, the desired location for the tool 15 is the next trajectory point, unless the trajectory data indicates that the tool should remain at the current trajectory point, such as where a microneedle tool is used and must go through a local area treatment cycle before moving on. When that next trajectory point is reached, the next point after that becomes the desired location of the tool, and the robot arm moves the tool toward that point, and so on, until the last point is reached.

The speed of the movement is regulated by the duration set out by the surgeon for the movement in the trajectory. Generally, the robot will move at a speed and accuracy that allows the tool to arrive at the points on schedule according to the specified duration. However, if the tool does not have sufficient time to get to the next point before it would be scheduled to leave for the point after that, the next point will be loaded as the desired location for the tool even if there was some error in the tool reaching the earlier point. Specifically, the robot arm will not dither trying to move to the exact location specified where the tool is behind schedule to leave for the next point in the trajectory. This results in some error in the movement along the trajectory. If the errors begin to appear significant, the surgeon can slow down the speed of the treatment to improve the accuracy of the system.

In addition to the procedure of going from point to point between the sequential trajectory locations, the navigation system also determines, based on the navigation unit sensor data, whether the tool 15 is at the proper distance from the patient and at the correct attitudinal angle, and incorporates that determination in the data defining where the tool should be, i.e., the desired location and orientation of the tool in Cartesian coordinates. This use of a desired location and orientation of the tool 15 as continually verified and required by the sensor apparatus 19 of the navigation unit 11 results in movement of the tool 15 in real operation essentially flying above the surface of the skin of the patient at a constant height and attitude as it proceeds between points on the trajectory.

Data defining the desired tool position and orientation is transferred in the Inverse Kinetics module 100. Inverse Kinetics module 100 converts the Cartesian coordinate data to desired values for the robot-arm joint angles Q, their velocities and accelerations, as was the case in the simulation. Those Q-data values are compared with current values for those parameters at software-implemented comparator 107, and the resulting difference is transmitted to CTC module 108. CTC module 108 then converts the resulting differences in Q values, velocities and accelerations to torques to be applied to the robot arm joints. That data defining torque values is then transmitted as electrical signals to the robot arm control system 23, which causes the motors of the arm to apply the indicated torques and move the arm 5.

The robot arm control system 23 also detects or receives from the arm 5 and transmits data defining, as Q values, the angles of rotation (Q_{act}), the velocity of rotation (Qd_{act}), and the acceleration of the rotations of each of the joints of the robot arm (Qdd_{act}). Those values are returned to comparator 107 for comparison with the next data from Inverse Kinetics module 100. When compared, the resulting difference is again sent to the CTC module to be converted into torque commands for the individual joint motors of the robot arm.

The Q_{act}, Qd_{act}, Qdd_{act} values are also sent to Forward Kinematics module 117, which converts them to Cartesian coordinates and direction vectors. Those coordinates are used to determine whether the tool has reached or is in a desired specified location of the current trajectory point. As mentioned above, once the feedback from the robot arm indicates that the current desired trajectory point location has been reached, the navigation system 121 loads the next trajectory point as the desired point, provided that no data in the trajectory data indicates a delay for treatment at the current point is required. The navigation software then initiates movement of the tool to the next trajectory point as the desired location, giving the Inverse Kinematics that Cartesian-coordinate location to start the robot arm moving the tool to that location.

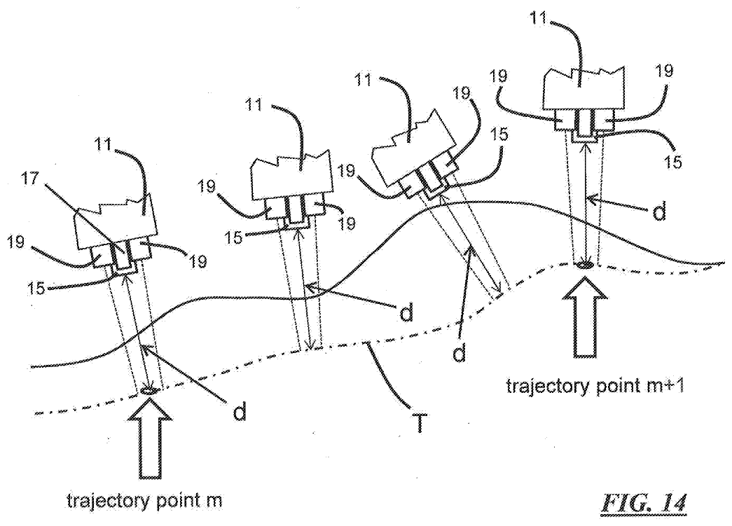

As described above, as the tool is moved, the sensors of the navigation unit continuously or continually provide sensor data of the distance and orientation of the tool from the skin of the patient, and that data is used by the navigation system 121 to control the distance and the angle of the tool at all times through the trajectory including the intervals between defined points of the trajectory. An example of this movement is illustrated in FIG. 14, which is a diagram showing an example of sequential positions of the tool 15 and navigation unit 11 between two points of the trajectory, m and m+1.

At point m, the sensors 47 of navigation unit 11 detect the orientation and distance of the tool 15 from the skin surface of the patient. The tool 15 at this point m is at a specified operating distance to point m, and also is at a specified angle, here normal or perpendicular to the skin. This orientation is obtained by sensors sending back the distance data continuously to the control system 25, which defines the distance and orientation of the tool 15 relative to the skin. This data is converted to the Cartesian coordinate system of the robot arm and processed through the Inverse Kinematics and other controls so as to maintain these two parameters, i.e., distance and perpendicularity.

The line of the trajectory between the points is a straight line, but as it runs over the contour of the patient's skin, it can encounter variations in its otherwise straight path, as shown in FIG. 14. As the navigation unit 11 is moved by the supporting robot arm (not shown) along the trajectory surface path T, the sensors 47 detect the distance and orientation of the tool 15 from the skin of the patient and the navigation system 121 continuously maintains the distance and perpendicular angle as the tool moves to the next point m+1. This may result in some considerable angular changes as the surface varies, as exemplified by the third position of the navigation unit 11 in the diagram, which shows the rotation of the navigation unit 11 to maintain the perpendicularity of the tool operating direction to the skin, which at this location has a steep rise in its contour. It of course can be envisioned that in some parts of the body, the variations in the surface are quite substantial, such as, e.g., in the area of a nostril or on an ear. The system of the invention will nonetheless follow the path between the trajectory points, angulating the tool so that it is directed at the appropriate attitudinal angle to the surface being treated.

The loop process continues until the trajectory is complete or the operator stops the operation manually.

In the actual procedure, the video from the camera 17 on the navigation unit 11 is transmitted through to and displayed in the window 95 of the GUI labeled “Endoscopic View” in which the surgeon can see the area of the patient exposed to action by the End Effector in high definition video, as well as the tool 15 itself.

During the procedure, the movement of the End Effector is essentially autonomous, and the End Effector proceeds from the first trajectory point to the next, and then the next after that, and so on until the full trajectory is completed. The surgeon may specify that the End Effector should proceed through the trajectory points at a rate defined by the Speed Control indicated at 97, by which the End Effector remains at each point at a relatively longer or shorter time within a predetermined range of maximum and minimum time intervals between trajectory points, moving to the next trajectory point automatically as the specified time interval ends.

Alternatively, during the operation, the surgeon may become more manually involved in the operation, and can accelerate the procedure at any given point by pushing the virtual button 98 labeled “Next” in the GUI, which sends an electrical signal to the arm 5 to move the navigation unit End Effector to the next trajectory point. Analogously, the surgeon may also direct the arm 5 and End Effector to return to the immediately previous trajectory point to expand on the treatment applied by pressing the virtual button 99 labeled “Previous” in the GUI.

The surgeon also may become involved in manual control where the End Effector is imparting heat or energy to the patient's skin by cauterization, or where the tool 15 is plasma torch or microneedle device. In such a situation, the surgeon can manually turn off the energy supply and stop the administration of the heat or energy to the patient by pressing the virtual button 100 in the GUI labeled “Cauterizer: OFF”, which will immediately stop the application of heat or energy to the patient.

The speed of the operation also may be adjusted by a slide control. Modifying the position of the slide control causes the data defining the duration of the trajectory to change, resulting in a slower or faster movement of the tool.

The procedure may be ended in a non-emergency by pressing the virtual button 101 labeled STOP. If the procedure is to be restarted, that can be achieved by pressing the virtual button 102 labeled “Repeat Procedure”. To return to the place where the procedure was stopped, the surgeon can press the Next button 98 until the End Effector is moved to the trajectory point at which the process was stopped previously.

When there is an emergency need to stop the process that may be done more immediately by pressing the virtual button 103 labeled “Emergency Stop”, which will stop everything in the system immediately, and possibly may take additional action of an emergency nature.

In the normal course of events, however, the procedure will finish autonomously, and the surgeon may then, if satisfied with the results, stand down the system by pressing the virtual button 105 labeled “Procedure Complete” which appropriately shuts down the system and retracts the arm 5 away from the patient.

An aspect of the invention that is particularly of importance is the control of movement of the End Effector on the robot arm 5 using the relative position and angulation data from the sensors in the navigation unit. The maintenance of the relative position to the patient is more important than, for example, the precise Cartesian coordinate location of the End Effector relative to the stationary base of the system. Being in the correct location and angulation relative to the skin surface of the patient means that slight movements of the patient do not affect the procedure being performed, because the relative position is maintained by the system.

The foregoing description relates to a generally autonomous procedure, in which a trajectory is laid in by a local or remote surgeon or user, and the system essentially autonomously implements the trajectory, together with the navigation unit maintaining the distance and attitude of the tool to the patient.

The system may also be employed where a surgeon remote from the patient directly controls by hand the movement of the medical tool on the robot arm by direct commands send electronically to the robot arm. There, the commands to move the tool through the trajectory in the previous embodiment are replaced by the commands of the remote surgeon to move the tool as he or she directs. The navigation unit and the associated navigation system 121 nonetheless continue to operate the sensors and to maintain, despite any manual commands from the surgeon, the distance and orientation of the tool at all times with respect to the patient.

That use of the navigation unit 11 is helpful, in that the commands of the remote surgeon can tend to introduce errors in the movement of the tool due to human error, or, as is even more likely, simply due to latency in the communications from a remote location, which might be as much as a few seconds, making precise control of the distance and orientation of the tool without the navigation unit difficult, even for an expert. Applying the navigation unit and navigation control loop of the present invention in that situation avoids some of the potentially negative aspects of such a remote control system.

While an embodiment with one robotic arm and a single tool has been shown here, it will be understood that an operating theater may employ two or more robotic arms with respective tools that may be different or even complementary to each other. However, each robot arm of a multi-arm system should have a respective navigation unit on it supporting the associated tool and maintaining its operative distance and orientation from the patient at all times.

The terms herein should be read as terms of description not limitation, as those of skill in the art with this disclosure before them will be able to make changes and modifications therein without departing from the spirit of the invention.

1. A robotic system for treating the skin of a patient, said system comprising:
a mechanical support device having a support portion, said mechanical support device supporting the support portion in a three-dimensional space of three-dimensional locations and in a range of three-dimensional angular orientations, said mechanical support device being configured to move the support portion in the three-dimensional space and over the range of angulations responsive to electronic control; and
a medical tool supported on the support portion so as to move with the support portion of the mechanical support device, said medical tool having an operative portion directed in an operative direction and configured to interact with the skin of the patient;
a sensor apparatus supported in a fixed position relative to the medical tool, said sensor apparatus sensing the skin of the patient and generating sensor electrical signals indicative of a distance and orientation of the operative portion of the tool relative to a part of the skin of the patient with which the tool is interacting; and
a navigation system directing movement of the medical tool via control of movement of the mechanical support device;
the navigation system receiving the sensor electrical signals and based thereon controlling the mechanical support device so as to maintain the operative portion of the tool at a predetermined distance from the skin of the patient and so as to maintain the operative portion of the tool at a predetermined angular orientation relative to the skin of the patient during movement of the mechanical support device.

2. The robotic system of claim 1, wherein the mechanical support device is a robotic arm made up of segments connected from a proximal end thereof to a distal end thereof.

3. The robotic system of claim 1, wherein the medical tool and the sensor apparatus are supported in a housing of a navigation unit fixedly supported on the support portion, and wherein the sensor apparatus comprises three sensor units supported in the housing, each of said sensor units detecting a respective distance thereof from the skin of the patient and producing a sensor electrical signal indicative of said distance, the navigation unit transmitting to the navigation system the three sensor electrical signals or a fourth electrical signal derived from the three sensor signals and indicative of the distance and orientation of the medical tool relative to the patient.

4. The robotic system of claim 1, wherein the navigation system has data defining a sequence of locations on or adjacent the patient, and the navigation system transmits commands to the mechanical support device that cause the mechanical support device to move the support portion and the medical tool to said locations in sequence, said navigation system sending commands that maintain the predetermined distance and orientation of the medical tool relative to the patient throughout movement thereof between the locations.

5. The robotic system of claim 4, wherein the data defines the locations in a Cartesian coordinate system and the navigation system uses data derived from the sensor signals defining a desired position of the medical tool in Cartesian coordinates, and wherein the navigation system performs an inverse kinematics determination in a control loop so as to determine desired positions of the segments of the robotic arm so as to place the medical tool in the desired position.

6. The robotic system of claim 1, wherein the support portion is on the distal end of the robotic arm, wherein the robotic arm has at least six degrees of freedom provided by relative rotation of the segments to each other and an accuracy of movement of the distal end and the support portion that is within a tolerance that is no more than 0.009 inches, and wherein the rotation of the segments is about joints therebetween at a speed of at least 180 degrees per second.

7. The robotic system of claim 1, wherein the medical tool is selected from the group consisting of a scalpel, scissors, and an electrocauterizer.

8. The robotic system of 1 claim, wherein the medical tool is a microneedle skin-treatment tool with an array of movable microneedles configured to be inserted into the skin of a patient.

9. The robotic system of claim 1, wherein the medical tool is a gas plasma skin treatment tool.

10. The robotic system of claim 1, wherein the system has
a high definition video camera supported adjacent the tool, said camera being directed toward the treatment area of the tool and transmitting video thereof; and
a user console with a display displaying the video to the user.

11. The robotic system of o claim 3, wherein the three sensor units of the sensor apparatus are supported rotatively distributed around the medical tool about an axis of the operative direction thereof, the sensor units each including a laser system detecting the respective distance of the sensor unit to skin of the patient.

12. The robotic system of claim 4, wherein the sequence of locations defines a trajectory and a duration of time within which the medical tool is to travel through said sequence of locations.

13. The robotic system of claim 1, wherein the navigation system controls movement of the robotic arm based on manually entered command signals received from a user at a remote location, and the navigation system moves the robotic arm so as to maintain the distance and orientation of the medical tool with respect to the patient's skin irrespective of any commands from the remote user that conflict therewith.

14. The robotic system of claim 1, wherein the predetermined orientation is normal to the skin of the patient in the operative area of the medical tool.

15. A method for treating a skin region of a patient, said method comprising:
scanning the skin region of the patient so as to derive three-dimensional data defining a surface contour of the skin region;
determining a sequence of points on the skin region at which treatment is to be applied;
providing a robotic apparatus movably supporting a skin treatment tool in a range of positions and angular orientations responsive to electrical control signals, said skin treatment tool having a sensor apparatus supported fixedly with respect thereto so as to move therewith;
performing the treatment of the skin region with the skin treatment tool, wherein the skin treatment tool is moved to the series of points by the robotic apparatus, and wherein, in each of the locations, an operative effect of the tool is directed to a respective location that corresponds to a respective one of the points;
sensing continually using the sensor apparatus during the treatment physical parameters defining a distance and orientation of the tool relative to the skin region of the patient, wherein the sensor apparatus generates electrical signals from which said relative distance and orientation are determined; and
controlling movement of the robotic apparatus based on the electrical signals so that, at each of the series of locations and throughout travel of the tool therebetween, the skin treatment tool is located and oriented at a predetermined distance and an predetermined angulation relative to the skin region.

16. The method according to claim 15, wherein the method further comprises
performing a simulation of the treatment prior to performing the treatment using the sequence of points, and
displaying a video of the simulation to a user, and
responsive to the user approval of the sequence of points of the simulation, performing the treatment with the tool being moved in sequence through locations corresponding to the sequence of points.

17. The method according to claim 16, wherein the robotic apparatus is a robotic arm made up of segments connected from a proximal end thereof to a distal end thereof, wherein the support portion is on the distal end of the robotic arm, wherein the robotic arm has at least six degrees of freedom provided by relative rotation of the segments to each other and an accuracy of movement of the distal end and the support portion that is within a tolerance that is no more than 0.009 inches, and wherein the rotation of the segments is about joints therebetween at a speed of at least 180 degrees per second.

18. The method according to claim 16, wherein the skin treatment tool is selected from the group consisting of a scalpel, scissors, and an electrocauterizer.

19. The method according to claim 16, wherein the skin treatment tool is a microneedle skin-treatment tool with an array of movable microneedles configured to be inserted into the skin of a patient, said microneedle tool being supported in a unit that when activated extends the microneedle tool forward out of the unit so as to engage the skin of the patient.

20. The method according to claim 16, wherein the skin treatment tool is a gas plasma skin treatment tool.

21. The method according to claim 16, wherein the method further comprises providing a high definition video camera supported adjacent the skin treatment tool, said camera being directed toward the treatment area of the skin treatment tool, and transmitting video of the treatment area to a user console with a display displaying the video to the user.

22. The method according to claim 16, wherein the sensor apparatus comprises three sensor units supported distributed around the tool equally around an axis of the operative direction of the tool, the sensor units each including a laser system detecting a respective distance of the sensor unit to skin of the patient.

23. The method according to claim 22, wherein the controlling of the robotic apparatus includes using a computer applying inverse kinematics to data derived from the sensor units of the sensor apparatus so as to derive data for desired rotational positions of parts of the robotic apparatus, and determining therefrom torque commands sent to the robotic apparatus using a control loop.

24. The method according to claim 16, wherein the sequence of points is determined from a historical sequence of points stored in a computer-accessible library of historical procedures.

25. A navigational unit comprising:
a housing configured to be secured to an end of a robotic arm, said housing supporting therein
a medical tool configured to provide therapeutic treatment to an area of skin of a patient positioned in an operative area located in an operative direction from the medical tool;
a camera supported fixedly adjacent the medical tool and deriving video of the operative area of the medical tool, said camera transmitting the video as an electrical video signal;
three laser-based distance sensors supported distributed around the medical tool, each of the sensor units continually detecting a distance from the sensor unit to the skin of the patient and transmitting sensor electrical signals containing data indicative of the respective distance, and
navigation electronics receiving the sensor electrical signals and the video and having an electrical connection over which the electronics transmit the video signal and an electrical data signal derived from the sensor electrical signals from which the distance and orientation of the medical tool relative to the patient can be determined.
